(19) United States
(12) Patent Application Publication
(10) Pub. No.: US 2019/0184005 A1
(43) Pub. Date: Jun. 20, 2019

(54) METHOD AND COMPOSITION FOR AMELIORATING AND ABATING SYMPTOMS RESULTING FROM RHEUMATOID ARTHRITIS, FIBROMYALGIA, AND CHRONIC PAIN OF UNKNOWN ORIGIN

(71) Applicant: HONOR C.W. M.D., LLC, Avalon, CA (US)

(72) Inventor: George Nelson, Woodland Hills, CA (US)

(73) Assignee: HONOR C.W. M.D., LLC, Avalon, CA (US)

(21) Appl. No.: 16/284,738

(22) Filed: Feb. 25, 2019

Related U.S. Application Data
(63) Continuation of application No. 15/683,164, filed on Aug. 22, 2017.
Application No. 15/683,164 is a continuation of application No. 12/759,620, filed on Apr. 13, 2010, now abandoned.
Application No. 12/759,620 is a continuation-in-part of application No. 12/426,838, filed on Apr. 20, 2009, now abandoned.
Application No. 12/426,838 is a continuation-in-part of application No. 12/298,904, filed on Oct. 28, 2008, now abandoned.
Application No. 12/298,904 is a continuation-in-part of application No. PCT/US2008/117755, filed on Oct. 14, 2008.
Application No. PCT/US2008/117755 is a continuation-in-part of application No. PCT/US2008/011233, filed on Sep. 26, 2000.

(51) Int. Cl. A61K 39/13; A61K 39/12

(52) U.S. Cl. CPC A61K 39/13; A61K 2039/58; A61K 39/12

(57) ABSTRACT

The present invention provides a composition and method for treating diseases associated with demyelination of the nerves, such as ALS, RA, Tremors/Parkinson's Disease, and MS, Alzheimer's disease, ALS, Guillain-Barre syndrome, atherosclerosis, schizophrenia, Tremors/Parkinson's disease, senile dementia, Muscular Dystrophy, Attention Deficit Disorder, Attention Deficit Hyperactivity Disorder, Complex Regional Pain Syndrome, Diabetes, Neuropathic Pain, Spider Arthritis West Nile Virus, Fibromyalgia, Shingles, Gout, Migraine Headaches, Post Polio Syndrome, Central Virus Deafness, Asthma, Chronic Pain Of Unknown Origin and Hepatitis C and for treating non-viral based cancers. By administering measured doses of an immunity-provoking agent and a bacterial antigen activator, patients suffering from ALS, RA, MS, Tremors/Parkinson's Disease, and prostate cancer and others realized immediate beneficial results with no side effects.

### CROSS REFERENCE TO RELATED PATENT APPLICATIONS

This nonprovisional patent application is a continuation of U.S. patent application Ser. No. 15/683,164, entitled Method For Ameliorating and Abating Symptoms Resulting From Rheumatoid Arthritis, Fibromyalgia, and Chronic Pain Of Unknown Origin, filed on Aug. 22, 2017. U.S. patent application Ser. No. 15/683,164 is a continuation of U.S. patent application Ser. No. 12/759,620, entitled Method For Ameliorating and Abating Symptoms Resulting From Rheumatoid Arthritis, Fibromyalgia, and Chronic Pain Of Unknown Origin, filed on Apr. 13, 2010; which is a continuation in part of U.S. patent application Ser. No. 12/426,838, entitled Process For Treatment Of Amyotrophic Lateral Sclerosis, Rheumatoid Arthritis, Tremors/Parkinson's Disease, Multiple Sclerosis, and Non-Viral Based Cancers, filed Apr. 20, 2009; which is a continuation in part of U.S. patent application Ser. No. 12/298,904, entitled “Process for Treatment of Rheumatoid Arthritis, Tremors/Parkinson's Disease, Multiple Sclerosis and Non-Viral Based Cancers,” filed on Oct. 28, 2008; which is the U.S. National Phase application of International Application Serial No. PCT/US08/11775, filed Oct. 14, 2008; which is a continuation in part of International Application Serial No. PCT/US08/11233, entitled Treatment for Rheumatoid Arthritis and Multiple Sclerosis filed Sep. 26, 2008; each of which is incorporated by reference in its entirety, and to which priority is claimed.

### FIELD OF USE

The present invention relates generally to the treatment of autoimmune disorders, and specifically, to the treatment of demyelinating diseases such as amyotrophic lateral sclerosis, rheumatoid arthritis, Tremors/Parkinson's Disease, multiple sclerosis, Alzheimer's Disease, Muscular Dystrophy, Attention Deficit Disorder, Attention Deficit Hyperactivity Disorder, Complex Regional Pain Syndrome, Diabetes, Neuropathic Pain, Spider Arthritis, West Nile Virus, Fibromyalgia, Shingles, Gout, Migraine Headaches, Senile Dementia, Post Polio Syndrome, Central Virus Deafness, Chronic Pain Of Unknown Origin, Asthma and Hepatitis C. The present invention also relates to the treatment of non-viral based cancers.

### BACKGROUND

In Rheumatoid arthritis (“RA”) is an autoimmune disease that is typically manifest by inflammation of the synovial joints. The development of RA progresses chronically, alternating between remission and relapse. Damage and deformation of joints can occur rapidly, particularly if the disease is untreated. As the disease progresses, RA can cause joint destruction, functional disability and premature mortality. RA can also include systemic inflammatory disease affecting multiple organs. RA patients often suffer physically and mentally from heavy pain all their lives. The cause of RA is presently unknown.

As an autoimmune disease, RA is characterized by a defect in the body's ability to distinguish foreign molecules from its own. The immune system attacks the synovial membrane, causing inflammation due to the infiltration of the membrane with T cells, plasma cells and macrophages. Formation of granulation tissue at the edges of the synovial lining is marked by extensive angiogenesis and enzyme production. These effects in turn cause progressive, erosive disintegration of adjacent cartilage and bone. In conjunction with the inflammation of the membranes, patients suffering from RA can also exhibit nerve abnormalities that primarily seem to involve segmental destruction of the myelin sheath.

Early stage prior art treatments typically attempt to ameliorate the pain symptoms through administration of non-steroidal anti-inflammatory drugs (NSAID). However, these treatments do little or nothing to affect the progression of RA.

Once a definitive RA diagnosis is made, conventional treatments include the use of steroids in conjunction with physical therapy and, if joint damage occurs, surgery. Again, these treatments have significant drawbacks and do not address the underlying causes of RA. For example, steroid therapy is associated with a number of well-known adverse side effects.

Specific compounds known as disease modifying anti-rheumatic drugs (DMARD) have been developed in an attempt to directly target the processes associated with RA. These DMARDs are typically administered in conjunction with NSATDs. Examples of such compounds include Remicade®, methotrexate, and Humira®, which are all immunomodulators designed to inhibit the function of the body's immune system. While such treatments can slow the attack of RA, they undermine the ability of the immune system to respond normally to infections and leave the patient vulnerable to other diseases. Furthermore, they do not address the underlying causes of RA. Moreover, there are potentially severe side effects from using these immomodulators and there are restrictions placed on users to avoid exercise, alcohol and to be concerned about drug interferences.

As no cure for RA exists, there exists a need for treatments that alleviate the pain and inflammation associated with RA without the drawbacks inherent in prior art strategies. Similarly, there is a need for treatments that mitigate the joint damage associated with RA. One object of the current invention is to provide such treatments while minimizing the negative effects on a patient's immune system.

In addition to RA, there are a number of other progressive or degenerative diseases, such as Crohn's disease, multiple sclerosis (“MS”), Tremors/Parkinson's Disease, Alzheimer's disease, amyotrophic lateral sclerosis (“ALS”), Guillain-Barre syndrome, atherosclerosis, schizophrenia, Parkinson's disease, senile dementia and others, associated with nerve damage. Although distinct, these diseases share common elements. Specifically, the precise origin or cause of these diseases remains unknown, yet they all exhibit damage to the nerves in the form of demyelination. As with RA, there is currently no cure for these diseases and prior art treatments have focused on modulating the patient's immune system. For example, Copaxone® is administered to patients suffering from MS in order to suppress immune response. Naturally, a significant side effect of such treatments is the potential for the patient to have a compromised immune system.

Accordingly, there exists a need for treatments for MS, Alzheimer's disease, Parkinson's disease and the like that minimize the drawbacks associated with the prior art. Similarly, there is a need for a treatment for such diseases that helps prevent demyelination.

In certain cancers, there may be a latent viral infection that remains quiescent until some signal triggers a release from latency. Once triggered, the tumorous cell begins to replicate. The identification or disease etiology is difficult to assign because in some infections, the DNA of the causation virus is integrated into the genome of the host cell and is transmitted vertically. It therefore behaves as a genetic attribute. In other circumstances, the causative microbe triggers the cancer-disease process and then disappears from the body and is no longer detectable. What is needed, therefore is a vaccine that prevents single strand linear viruses from triggering the release of cancer from latency. It is these types of cancers, such as e.g., prostrate, liver, pancreatic, and lung cancer, that are referred to as the non-viral based cancers. Non-viral based cancers are to be contrasted with viral cancers whose etiology has been directly traced to viral causes. At present, only two viruses, human T-cell lymphotropic virus and human papillomavirus, are considered to be human tumor viruses. However, several other candidate viruses are implicated by epidemiological correlation, by serologic relationship or by recovery of virus from tumor cells.

The present invention satisfies these and other needs.

### SUMMARY

The present invention is directed to composition useful in treating symptoms of diseases associated with demyelination of the nerves, such as ALS, RA, MS, Tremors/Parkinson's Disease, non-viral based cancers, Alzheimer's Disease, Muscular Dystrophy, Attention Deficit Disorder, Attention Deficit Hyperactivity Disorder, Complex Regional Pain Syndrome, Diabetes, Neuropathic Pain, Spider Arthritis, West Nile Virus, Fibromyalgia, Shingles, Gout, Migraine Headaches, Senile Dementia, Post Polio Syndrome, Central Virus Deafness, Asthma, Chronic Pain Of Unknown Origin and Hepatitis C. In one embodiment of the invention, the composition includes an immunity-provoking agent and a bacterial antigen activator. Preferably, the immunity-provoking agent is a vaccine for a single-stranded RNA virus and more preferably, the immunity-provoking agent is an inactivated polio vaccine. Also preferably, the bacterial antigen activator is either tetanus toxoid, typhim VI, diphtheria toxoid or mixtures thereof

Preferably, the composition comprises 5 parts of the inactivated polio vaccine to 1 part of the tetanus toxoid and 1 part of the typhim VI. Alternatively, the composition comprises 5 parts of the inactivated polio vaccine to 2 parts of either tetanus toxoid, typhim VI, or diphtheria toxoid.

Also preferably, the composition is formulated for subcutaneous injection.

Another aspect of the invention is directed to a method for treating pain and inflammation in a patient with one or more of the demyelinating diseases comprising the steps of preparing a composition of an immunity-provoking agent and a bacterial antigen activator; and administering the composition to the patient. Preferably, the step of administering the composition comprises administering the composition subcutaneously. More preferably, the step of administering the composition comprises administering approximately 0.70 cc of the composition.

In one embodiment, the method includes treating a patient suffering from a demyelinating disease. Examples of such diseases include rheumatoid arthritis, multiple sclerosis, Alzheimer's disease, ALS, Guillain-Barre syndrome, atherosclerosis, schizophrenia, Tremors/Parkinson's disease, senile dementia, Alzheimer's Disease, Muscular Dystrophy, Attention Deficit Disorder, Attention Deficit Hyperactivity Disorder, Complex Regional Pain Syndrome, Diabetes, Neuropathic Pain, Spider Arthritis West Nile Virus, Fibromyalgia, Shingles, Gout, Migraine Headaches, Senile Dementia, Post Polio Syndrome, Central Virus Deafness, Asthma, Chronic Pain Of Unknown Origin and Hepatitis C.

In another embodiment, the method includes treating a patient suffering from a non-viral based cancer disease. Examples of such cancer diseases include prostrate cancers. The treatment of these disease conditions according to the compositions and methods of the invention eliminate the restrictions placed on the users of prior art immunomodulators and the potentially severe side effects of these compounds.

One embodiment of the present disclosure may be a composition comprising: (a) 5 parts by volume of an inactivated polio vaccine and (b) 2 parts by volume of a bacterial antigen activator. The bacterial antigen activator may be selected from the group of bacterial antigen activators consisting of one or more of: tetanus toxoid; typhim VI; diphtheria toxoid; and combinations thereof. Alternatively, the bacterial antigen activator may comprise 1 part by volume tetanus toxoid and 1 part by volume typhim VI. The composition may be packaged for subcutaneous injection. The composition may be configured to be used to treat diseases selected from the group of diseases selected from one or more of: rheumatoid arthritis; multiple sclerosis; Alzheimer's disease; ALS; Guillain-Barre syndrome; atherosclerosis; schizophrenia; Tremors/Parkinsons's disease; senile dementia; Muscular Dystrophy; Attention Deficit Disorder; Attention Deficit Hyperactivity Disorder; Complex Regional Pain Syndrome; Diabetes; Neuropathic Pain; Spider Arthritis; West Nile Virus; Fibromyalgia, Shingles; Gout; Migraine Headaches; Post Polio Syndrome; Central Virus Deafness; Asthma; Chronic Pain Of Unknown Origin; Hepatitis C; and combinations thereof.

On embodiment may be a dosage preparation kit comprising: at least one 70 milliliter dosage of a composition, wherein the composition may comprise 5 parts by volume of an inactivated polio vaccine and 2 parts by volume of a bacterial antigen activator. The bacterial antigen activator is selected from the group of bacterial antigen activators consisting of one or more of: tetanus toxoid; typhim IV; diphtheria toxoid; and combinations thereof. In one embodiment may be a bacterial antigen activator comprises 1 part by volume tetanus toxoid and 1 part by volume typhim VI. The composition may be packaged for subcutaneous injection. The composition is configured to be used to treat diseases selected from the group of diseases selected from one or more of: rheumatoid arthritis; multiple sclerosis; Alzheimer's disease; ALS; Guillain-Barre syndrome; atherosclerosis; schizophrenia; Tremors/Parkinsons's disease; senile dementia; Muscular Dystrophy; Attention Deficit Disorder; Attention Deficit Hyperactivity Disorder; Complex Regional Pain Syndrome; Diabetes; Neuropathic Pain; Spider Arthritis; West Nile Virus; Fibromyalgia, Shingles; Gout; Migraine Headaches; Post Polio Syndrome; Central Virus Deafness; Asthma; Chronic Pain Of Unknown Origin; Hepatitis C; and combinations thereof

Another embodiment may be a composition comprising: (a) 5 parts by volume of an inactivated polio vaccine; and (b) 2 parts by volume of a bacterial antigen activator; wherein the bacterial antigen activator comprises 1 part by volume tetanus toxoid and 1 part by volume typhim VI; wherein the composition is packaged for subcutaneous injection; and wherein the composition is configured to be used to treat diseases selected from the group of diseases selected from one or more of: rheumatoid arthritis; multiple sclerosis; Alzheimer's disease; ALS; Guillain-Barre syndrome; atherosclerosis; schizophrenia; Tremors/Parkinsons's disease; senile dementia; Muscular Dystrophy; Attention Deficit Disorder; Attention Deficit Hyperactivity Disorder; Complex Regional Pain Syndrome; Diabetes; Neuropathic Pain; Spider Arthritis; West Nile Virus; Fibromyalgia, Shingles; Gout; Migraine Headaches; Post Polio Syndrome; Central Virus Deafness; Asthma; Chronic Pain Of Unknown Origin; Hepatitis C; and combinations thereof.

### DETAILED DESCRIPTION

The present invention is a process for treating diseases associated with demyelination of the nerves, such as RA, MS, Alzheimer's disease, ALS, Guillain-Barre syndrome, atherosclerosis, schizophrenia, Tremors/Parkinson's disease, senile dementia, for treating non-viral based cancers, Alzheimer's Disease, Muscular Dystrophy, Attention Deficit Disorder, Attention Deficit Hyperactivity Disorder, Complex Regional Pain Syndrome, Diabetes, Neuropathic Pain, Spider Arthritis, West Nile Virus, Fibromyalgia, Shingles, Gout, Migraine Headaches, Senile Dementia, Post Polio Syndrome, Central Virus Deafness, Chronic Pain Of Unknown Origin, Asthma and Hepatitis C. By administering measured doses of an immunity-provoking agent and a bacterial antigen activator, patients suffering from these diseases and cancers have realized beneficial results. In connection with the non-viral based cancer diseases, the vaccination should be used, as appropriate, along with surgery, radiation and chemotherapy. However, as a vaccine, the present invention has the ability to combat the genesis of the non-viral cancer disease.

As discussed above, there exists a significant class of diseases for which the causative agents are poorly understood, but share a common symptom of nerve damage due to demyelination.

Myelin is the protective sheath around axons in the nervous system, also known as “white matter.” Myelin insulates the nerve and facilitates the conduction of the electrical potential associated with a neuronal signal. The myelin sheath is composed of glycolipids and proteins deposited around the axon by glial cells. Myelination of the nerves is an ongoing process that occurs during development and throughout childhood.

Demyelination can occur when the patient's immune system attacks the sheath, removing portions of the myelin from the axon. The physiological response to this damage causes the formation of gliotic plaques that interfere with conduction of the nerve impulses.

Without being limited to a particular theory, it is proposed that viral infection causes the patient's myelin to become targeted by the immune system. In response to the infection, the immune system produces antibodies to antigens associated with the infectious agent. However, when these antibodies are insufficiently specific and also recognize normal host antigens, such as components of the myelin sheath, a destructive, autoimmune response can result. Specifically, a dormant childhood infection could form the basis for a subsequent immune response that leads to one of the noted neurodegenerative diseases. Triggers for such a response could be severe physical/psychological trauma or it could be exposure to a suitable antigen or even the natural completion of the myelination process during the transition into adulthood.

In a related modality, a dormant childhood infection can also form the basis for triggering the replication of cancerous cells that have been in a latent state. Accordingly, treatment with a suitable vaccine should counter this effect and compositions of the invention include an immunity-provoking agent.

Suitable immunity-provoking agents are preparations, such as vaccines, having the ability to confer a degree of immunity to a patient for a demyelinating disease. Preferably, the disease is also known to have the ability to penetrate the central nervous system (“CNS”) of the patient.

In one embodiment of the invention, the immunity-provoking agent comprises a polio vaccine. Poliomyelitis is a disease characterized by degradation of the myelin sheath, often leading to paralysis. The polio virus is a human enterovirus and member of the family of Picornaviridae composed of a single-stranded positive-sense RNA genome and protein capsid. Although a majority of polio infections are asymptomatic, in a small percentage of cases the virus does invade the patient's CNS, leading to the nerve damage that is the primary symptom of the disease. More preferably, the immunity-provoking agent comprises inactivated polio vaccine (“IPV”), such as trivalent IPV.

Other suitable uses for this vaccine with the single stranded RNA-based viruses that may be used in the practice of the invention include vaccines for rubella, mumps, measles, Rhinovirus virus, hepatitis A virus, Hepatitis C virus, Yellow Fever Virus, Dengue Virus and West Nile Virus.

It has been found that the compositions of the invention also require a bacterial antigen activator in conjunction with the immunity-provoking agent. Suitable bacterial antigen activators include gram-negative bacteria vaccines and gram-positive bacteria vaccines. Specific bacterial antigen activators found to be useful in the practice of the invention include tetanus toxoid and typhoid vaccine.

Clostridium tetani is a gram-positive, obligate anaerobic bacterium that produces the neurotoxin tetanospasmin. Tetanus toxoid is a modified form of tetanospasmin shown to stimulate the production of suitable antibodies and confer an immunity to tetanus. Salmonella enterica serovar typhi is a gram-negative, flagellated, rod-shaped bacterium and is the disease agent in typhoid fever. Typhoid vaccines are prepared from antigens particular to the bacterium. For example, the typhim VI vaccine is prepared from a cell surface polysaccharide of S. typhi. Typhim Vi®, Typhoid Vi Polysaccharide Vaccine, produced by Sanofi Pasteur S A, for intramuscular use, is a sterile solution containing the cell surface Vi polysaccharide extracted from Salmonella enterica serovar Typhi, S typhi Ty2 strain. The organism is grown in a semi-synthetic medium without animal proteins. The capsular polysaccharide is precipitated from the concentrated culture supernatant by the addition of hexadecyltrimethylarnmonium bromide and the product is purified by differential centrifugation and precipitation. The potency of the purified polysaccharide is assessed by molecular size and 0-acetyl content. Phenol 0.25%, is added as a preservative. The vaccine contains residual polydimethylsiloxane or fatty-acid ester-based antifoam. The vaccine is a clear, colorless solution. Each dose of 0.5 ml is formulated to contain 25 μg of purified Vi polysaccharide in a colorless isotonic phosphate buffered saline (pH 7±0.3), 4.150 mg of Sodium Chloride, 0.065 mg of Disodium Phosphate, 0.023 mg of Monosodium Phosphate and 0.5 mL of Sterile Water for Injection.

The use of Diphtheria Toxoid to develop another vaccine to a single-strand virus is also intended to be within the scope of the invention.

Accordingly, in a presently preferred embodiment, the subject invention is directed to composition for subcutaneous injection comprising IPV, typhim VI and tetanus toxoid. More specifically, the composition of the invention preferably comprises 1 part tetanus toxoid, 1 part typhim VI, and 5 parts IPV. Alternatively, the composition comprises 2 parts tetanus toxoid and 5 parts IPV. In another alternative, the composition comprises 2 parts typhim VI and 5 parts IPV. In yet another alternative, diphtheria toxoid can be substituted for the tetanus toxoid or for the typhim VI and can be mixed with one or both. The above ratios are all based on concentrations of IPV at (80 D antigen units Type 1)/mL, (16 D antigen units Type 2)/mL, and (64 D antigen units Type 3)/mL, tetanus toxoid at 10 Lf (flocculation units)/mL and 2 units antitoxin/mL, and typhim VI at 50 mg/mL.

The frequency and size of the vaccine dosage can be increased or decreased according to the patient's physical stature, and the general nature of the patient's health. However, preferably, the dosage remains at 0.70 cc per treatment.

For treatment in a patient suffering from pain and inflammation, the invention is a method comprising the steps of preparing a composition of immunity-provoking agent and bacterial antigen activator and administering the composition to the patient.

Preferably, the methods of the invention are directed to treatment of symptoms associated with RA, MS, Alzheimer's disease, ALS, Guillain-Barre syndrome, atherosclerosis, schizophrenia, Parkinson's disease, senile dementia, Alzheimer's Disease, Muscular Dystrophy, Attention Deficit Disorder, Attention Deficit Hyperactivity Disorder, Complex Regional Pain Syndrome, Diabetes, Neuropathic Pain, Spider Arthritis, West Nile Virus, Fibromyalgia, Shingles, Gout, Migraine Headaches, Senile Dementia, Post Polio Syndrome, Central Virus Deafness, Chronic Pain Of Unknown Origin, Asthma and Hepatitis C and other diseases characterized by demyelization, and furthermore to the treatment of non-viral based cancers.

As noted above, the composition of the method preferably comprises 1 part tetanus toxoid, 1 part typhim VI, and 5 parts IPV, or 2 parts tetanus toxoid and 5 parts IPV, or 2 parts typhim VI and 5 parts IPV. In other embodiments, diphtheria toxoid can be substituted for some or all of the tetanus toxoid or typhim VI.

Also preferably, the step of administering the composition comprises subcutaneously injecting 0.70 cc of the composition.

With regard to subcutaneous administration, the epidermis is composed of 4-5 layers depending on the region of skin being considered. Those layers in descending order are the cornified layer (stratum corneum), clear/translucent layer (stratwn lucidum), granular layer (stratum granulosum), spinous layer (stratum spinosum), and basal/germinal layer (stratum hasa!elgerminativum). The term Malpighian layer (stratum malpighi) refers to both the basal and spinosum layers. When the composition is administered subcutaneously, in a preferred embodiment the syringe should be located in the first, second or third layers, or between first and second layers, or between second and third layers. Once located in these regions, the bolus of the composition is delivered.

A. Case Studies for Rheumatoid Arthritis (RA), Multiple Scleroses (MS), and Tremor's/Parkinson's (P), Prostrate Cancer (PC) and Amyotrophic Lateral Sclerosis (ALS).

Rheumatoid Arthritis (RA)

1. RA is a 61 year old male who has suffered from rheumatoid arthritis in his hands, fingers and back for the last 10 years. He started the medication 5 years ago and within one hour after taking the medication, the pain in his hands, fingers and back disappeared and by the second medication he continued to have no pain and no limitations of movement. He is basically symptom free of his rheumatoid arthritis and has continued taking the medication on a weekly basis. Absolutely no side effects.

2. RA is a 63 year old female who gave up golf as a result of rheumatoid arthritis. She has it in her hands, as well as her wrists and believes in her back for 10 years. She started the medication 2 years ago and within 45 minutes after the medication was administered, she was basically pain free, and had full and complete movement of both her wrists, hands and noticed no back pain whatsoever. She takes the medication once every 5 days and continues to remain pain free. Absolutely no side effects.

3. RA is a 82 year old man who had severe rheumatoid arthritis for 20 years. For the last 20 years both of his hands were clenched in a fist position and he suffered with severe pain in his hands. He received his first medication 3 years ago. After 45 minutes taking the medication he was crying for joy because this was the first time in 20 years he was without pain and an hour and a half after medication he was able to open his hands one inch. As his treatment continued every 5 days he regained full use of his hands with no pain and absolutely no side effects.

Multiple Scleroses (MS)

4. MS is a 62 year old female patient who has advanced MS. For eight years she suffered with severe pain in the right leg and was confined to a wheelchair, had incontinence, dysentery and multiple brain sheers (her doctor states that the last time she had seen a patient with this many brain sheers, it was a corpse). She started her medication 2 Y2 years ago. Her first medication reduced her pain by 50% and the 2nd medication 2 days later, within 45 minutes had no pain at all. The 3rd medication 4 days later she was still pain free and was able to stand and use a walker to help her get around. The 4th medication just 4 days later, she still showed no signs of pain, incontinence or dysentery and had no side effects. She began taking the medication every 5 days to maintain a healthy pain free life still with no dysentery and absolutely no side effects.

Tremor's/Parkinson's Disease (P)

5. P is a 64 year old man who noticed an occasional slight tremor in his left hand one year ago. He thought it was nerves. As time went on, the tremors were more frequent. He consulted with his doctor and was told it was it could be nerves or the beginning of Parkinson's Disease but there was no way to tell without an autopsy (not an option.) He tried compound vitamins, no help. After his first shot of the medication, the tremors stopped within 45 minutes, with no side effects. One week later, the left hand started some movement, I gave him another shot and the movement/tremors stopped. He has taken weekly shots since, and there have been no tremors and no side effects.

Prostrate Cancer

6. Twelve years ago P had a PSA score of 68 and a Gleason score of 7. A radical prostrate ectomy was performed, and P was given a prognosis of one to two years additional life. After P began administering the vaccination of the present invention, P's PSA score was −0.03 and has remained that way for twelve years.

Amyotrophic Lateral Sclerosis

7. YB is a patient that is in the final stages of ALS. She been on various pain medications over the years, but has not realized any significant pain abatement. Prior to receiving the vaccine, YB could not talk, her fingers were locked in a claw-like position, and she suffered from edema in her feet, legs, back and hand. Because of her pain, she was unable to move her jaw, thereby restricting her ability to eat. YB was also restricted from raising her arms above her chest due to the severe pain. In addition, YB suffered from shortness of breath, requiring an oxygen tank for breathing at night.

Vaccine was administered to YB four times per day. Within five days of continuous treatment, YB experienced significant reduction in her pain and edema. In addition, YB regained the ability to raise her arms and open her jaw. YB no longer needs an oxygen tank at night, and has regained the ability to speak.

B. Case Studies for Muscular Dystrophy, Post Polio Syndrome, West Nile Virus, Fibromyalgia, CRS, Attention Deficit/Hyperactivity Disorder, Neuropathic Pain, Diabetic Neuropathy, Multiple Sclerosis, Chronic Pain, Hepatitis C.

1. Complex Regional Pain Syndrome

LP, 55 year old female, was diagnosed with Reflex Sympathic Dystrophy also known as Complex Regional Pain Syndrome approximately seven years ago. Five years ago LP was also diagnosed with gout and shingles. About 10 months ago, LP became 100% deaf in right ear and 30% deaf in left ear, leading to a diagnosis of Viral Central deafness.

Within 20-30 minutes of administration of the treatment, the patient observed the following effects. Pressure and pain in legs were gone. Ankle and leg swelling were gone as was stabbing pain. Gout in big toe disappeared. Onset of shingles episode reversed. Extreme pressure in right ear relieved. Hearing in left ear clearer. Hearing ability went from monoaural to binaural. Slight hearing in right ear restored (5-10%). Neck pain and stiffness were gone, and LP had the restoration of complete range of motion in her neck; headache also relieved. Feet pain completely relieved, restoring ability to walk normally.

2. Muscular Dystrophy

Male age 42 with Muscular Dystrophy contracted the disease at age 16. Patient exhibited bad balance and difficulty walking without a cane. He experienced chronic pain in his legs, back and arms. Two hours after first treatment, the patient said that the pain was gone, the tightness in his legs and arms was gone and he could walk easily with no pain or other problems.

3. Post Polio Syndrome

Male age 79 with Post Polio Syndrome contracted polio at age 6, recovered and was doing fine until four years ago when he exhibited symptoms of Post Polio Syndrome, giving him weakening of the muscles, fatigue, pain in the muscles and joints, shortness of breath and sleeping problems. Within one hour of the treatment, the pain was gone. After two hours, he felt new energy and was walking easier and breathing better, with no side effects from the treatment.

4. West Nile Virus

Female age 35 with West Nile Virus complained of pain in her eyes, headaches and muscle and stomach pain. Within two hours of treatment, she was pain free and her condition continued to improve with no side effects.

5. Fibromyalgia

30 year old female with Fibromyalgia had pain throughout her body, aching and fatigue;

she experienced slight swelling of the muscles, headaches and numbness and tingling of the extremities. After her first treatment, she was pain free within one hour and continued to improve with no side effects.

6. Attention Deficit/Hyperactivity Disorder

Male patient age 59 diagnosed with AD/HD at age 55. Patient stopped AD/HD medications forty-eight hours before treatment. Within one hour of treatment, patient was observed to be calmer, which lasted for approximately seven days. Patient experienced an overall evenness in his nature and thoughts. Normal stress factors did not have usual negative result. Patient felt that he had his normal energy level without ups and downs associated with the AD/HD medications he had been taking for several years.

7. Neuropathic Pain of Mixed Etiology Including Diabetic Neuropathy, Multiple Sclerosis and Lumbar Radiculopathy

This involves utilizing two patients data together and meshing the information. The parties were middle aged males. One had a history of neuropathic pain extending from questionably some vitamin induced neurpathic pain or lack thereof of vitamins, lumbar radiopathy from disc bulges, facet hypertrophy, joint pain, and shooting sciatica pain down the legs. The other patient had nerve pain into the feet and leg bilaterally from multiple sclerosis and had undergone standard therapies with multiple sclerosis medications having serious side effects. He then underwent a month's treatment of the medication from Salubrious Pharmaceuticals LLC.

Treatment began with the single arm protocol and increase to dual arm protocol on a second visit and then increased the dosage on the third visit. Between the third and fourth visit the dose was maintained. Ultimately, the final dose was 80 IPV/40 B.A. per arm. Both patients did very well and had over 75% improvement in their pain function. The patient with radiculopathy from the disc bulge, facet hypertrophy and lack of vitamins had improved ability to walk, improved sensation of the ground with the foot as if he can curl his toes and feel where he was going. His balance improved and felt good throughout the entire process. His head was clear. He moved and walked better. He had no falls which had been an issue in the past. He had improved urinary continence and the ability to control bladder and bowel function.

The second patient had significant improvement in his bladder and bowel function. He did not have a significant effect on the nerve pain. However, patient was able to sleep better and overall felt much better. He had no feelings of flare-up or need for any types of muscular dystrophy medications. All of his medications were stopped for twelve weeks. There were no steroids given during that time and the patient felt good. He was able to get into a regimen of exercise on a daily program and overall was feeling better.

Both patients seem to respond favorably during the one month trial and there were no side effects noted.

8. Chronic Pain of Unknown Origin

PT is a 43 year old female with pain of unknown origin. She has multiple other medical comorbidities but has not clearly ascertained an underlying cause for her self-reported levels of pain. The pain is very severe which has necessitated high doses of opiates of which she has poorly responded. The pain has been in different areas and it rotates between the abdomen, back, neck, hips and joints. She has not had one particular protocol even any subtype RSD or fibromyalgia. A number of diagnostic codes have been used including fibromyalgia, causalgia/RSD, mononeuritis, radiculopathy, DOD, DID, associated myalgias, headaches, chronic fatigue syndrome and a number of other diagnostic codes to allow for us to try to work up some of her underlying issues but ultimately there has not been any clear cause for anything.

She came in for the trial and was given a double dose, one in each arm. She did very well and within one hour she felt the pain go away. She described it as when she was younger she had a migraine and was given Imitrex at the hospital (or some shot at the hospital which she believes was Imitrex now). Patient states that it felt like crackling. The pain crackled and went away very quickly. Within one hour she said the pain was decreased by over 75%. She had effect that lasted beyond one week. However, at nearly one week mark she said the pain started to come back more frequently and she started to develop a lot of the symptoms that she started in the beginning which were difficulty with sleep, some memory deficits and some other secondary side effects with agitation.

We are still awaiting a functional MRI on this patient and a PET scan to see if possibly there was ever a stroke. A central post pain syndrome has been of the potential ideas as her underlying cause. However, from the changes in the pair so frequently, there must have been an emotional component. She has had a lot of stress in her life. The most important aspect is that she had no side effects to the study drug from Salubrious Pharmaceuticals and had an amazing improvement during that week. At the end she asked how she could continue to get the medication because it changed her life so much.

9. Hepatitis C

Discussion with a female patient who has Hepatitis C and a number of other medical comorbidities that had a very aggressive history of drug abuse as a child and many other medical conditions that are not clear. The only etiological origin of her pain is she has cervical ODD, cervical DJD, and extreme myalgias. She had undergone trigger point injections, epidurals, nerve blocks, facet joint blocks, many other adjuvant therapies, chiropractic care, alternative medicine with some other practitioners, vitamin and mineral supplementation. She had contracted Hepatitis C while doing drugs in her youth but had a flare up of the hepatitis C approximately two years ago. She was treated with Interferon and Ribavirin and had weekly injections which made her very sick. She lost a lot of her energy and had very little ability to function. She ultimately needed to go on medical leave because of the amount of pain that she was having. She was asked to get viral loads and Hepatitis panels prior to testing. She was not able to afford it but went out and got some labs done. She was not sure where results were sent. Treating physician did not receive copies of lab reports.

Her first injection was at the normal dose in one arm. She noticed an improvement in her pain and felt as if things were melting inside of her. She felt uneasiness but within a short period of time she started to feel a little bit better. Within the next four to five days, she felt much better. Her pain medication was too strong for her and she was surprised how well it was working and actually needed to decrease it. She said she felt like the Salubrious Pharmaceuticals medication wore off on her and did not last long enough. It lasted five days or so and then started to wear off. She came back to the physician after the seven day mark and was given a follow up injection. This time she was given a double dose in each arm. She felt immediate response within one hour with significant improvement. Overall, it made significant improvement in the quality of her life.

10. Alzheimer's Disease

Female patient SN, aged 82, was diagnosed with Alzheimer's disease in 2006 after a very stressful life event. She had decreasing mental function, such that her husband stated that he needed to take steps to help her memory everywhere around the house. For example, white out was placed on the toaster over to help her know which buttons to push to cook. When treating physician met SN she could not recall her marriage date and multiple other facts about her life. One hour after giving SN the treatment, she was able to recall answers to questions that came from both short term and long term memory. The questions were asked once, and some of the questions were over materials that the treating physician had presented to her or asked her about one hour before administering treatment. SN's daughter and husband were present, and they observed that she had made an enormous memorable improvement. SN was smiling, happy, her demeanor was filled with joy and happiness. Treating physician was astonished at the speed of her response to the treatment and her improved ability to respond to the treating physician's questions. She was asked questions about her articles of clothing, the treating physician's recitation of the side effects and her wedding date, all of which she was able to answer after treatment.

11. Alzheimer's Disease

Male patient JM age 73 has been afflicted with Alzheimer's disease for several years. He has progressively lost his ability to reason, calculate and plan. He has been afflicted with hallucinations where he will talk to pictures on the wall as if communicating with the person pictured. Similarly, he will speak to persons on television in similar fashion.

JM received an injection of the treatment in each arm. Subsequent to receiving the treatment, after about an hour, JM was able to count backwards from 10 to 1, without much hesitation. This was an exercise he had wholly failed to perform before the treatment. Additionally, he managed some identification of events in his past such as date of birth, and memories from previous employment. He was observed to act in a more involved and coherent manner than in recent past. He was able to recall the specific details of conversations that had occurred 27 hours prior.

One will appreciate that in the description above and throughout, numerous specific details are set forth in order to provide a thorough understanding of the present invention. It will be evident, however, to one of ordinary skill in the art, that the present invention may be practiced without these specific details. In other instances, well-known structures and devices are shown in block diagram form to facilitate explanation. The description of the preferred embodiments is not intended to limit the scope of the claims appended hereto.

What is claimed is:

1. A composition comprising:
(a) 5 parts by volume of an inactivated polio vaccine; and
(b) 2 parts by volume of a bacterial antigen activator.

2. The composition of claims 1, wherein said bacterial antigen activator is selected from the group of bacterial antigen activators consisting of one or more of: tetanus toxoid; typhim VI;
diphtheria toxoid; and combinations thereof.

3. The composition of claim 1 wherein said bacterial antigen activator comprises 1 part by volume tetanus toxoid and 1 part by volume typhim VI.

4. The composition of claim 1, wherein said composition is packaged for subcutaneous injection.

5. The composition of claim 1, wherein said composition is configured to be used to treat diseases selected from the group of diseases selected from one or more of: rheumatoid arthritis; multiple sclerosis; Alzheimer's disease; ALS; Guillain-Barre syndrome; atherosclerosis; schizophrenia; Tremors/Parkinsons's disease; senile dementia; Muscular Dystrophy; Attention Deficit Disorder; Attention Deficit Hyperactivity Disorder; Complex Regional Pain Syndrome; Diabetes; Neuropathic Pain; Spider Arthritis; West Nile Virus; Fibromyalgia, Shingles; Gout; Migraine Headaches; Post Polio Syndrome; Central Virus Deafness; Asthma; Chronic Pain Of Unknown Origin; Hepatitis C; and combinations thereof.

6. A dosage preparation kit comprising:
at least one 70 milliliter dosage of a composition;
wherein said composition compriss 5 parts by volume of an inactivated polio vaccine and 2 parts by volume of a bacterial antigen activator.

7. The dosage preparation kit of claim 5, wherein said bacterial antigen activator is selected from the group of bacterial antigen activators consisting of one or more of: tetanus toxoid; typhim IV; diphtheria toxoid; and combinations thereof.

8. The dosage preparation kit of claim 5, wherein said bacterial antigen activator comprises 1 part by volume tetanus toxoid and 1 part by volume typhim VI.

9. The dosage preparation kit of claim 5, wherein said composition is packaged for subcutaneous injection.

10. The dosage preparation kit of claim 5, wherein said composition is configured to be used to treat diseases selected from the group of diseases selected from one or more of: rheumatoid arthritis; multiple sclerosis; Alzheimer's disease; ALS; Guillain-Barre syndrome; atherosclerosis; schizophrenia; Tremors/Parkinsons's disease; senile dementia; Muscular Dystrophy; Attention Deficit Disorder; Attention Deficit Hyperactivity Disorder; Complex Regional Pain Syndrome; Diabetes; Neuropathic Pain; Spider Arthritis; West Nile Virus; Fibromyalgia, Shingles; Gout; Migraine Headaches; Post Polio Syndrome; Central Virus Deafness; Asthma; Chronic Pain Of Unknown Origin; Hepatitis C; and combinations thereof.

11. A composition comprising:
(a) 5 parts by volume of an inactivated polio vaccine; and
(b) 2 parts by volume of a bacterial antigen activator;
wherein said bacterial antigen activator comprises 1 part by volume tetanus toxoid and 1 part by volume typhim VI;
wherein said composition is packaged for subcutaneous injection; and
wherein said composition is configured to be used to treat diseases selected from the group of diseases selected from one or more of: rheumatoid arthritis; multiple sclerosis; Alzheimer's disease; ALS; Guillain-Barre syndrome; atherosclerosis; schizophrenia; Tremors/Parkinsons's disease; senile dementia; Muscular Dystrophy; Attention Deficit Disorder; Attention Deficit Hyperactivity Disorder; Complex Regional Pain Syndrome; Diabetes; Neuropathic Pain; Spider Arthritis; West Nile Virus; Fibromyalgia, Shingles; Gout; Migraine Headaches; Post Polio Syndrome; Central Virus Deafness; Asthma; Chronic Pain Of Unknown Origin; Hepatitis C; and combinations thereof.
